(19) United States
(12) Patent Application Publication
(10) Pub. No.: US 2025/0288366 A1
(43) Pub. Date: Sep. 18, 2025

(54) Medical Sensing Devices and Systems

(71) Applicant: Bard Access Systems, Inc., Salt Lake City, UT (US)

(72) Inventors: Anthony K. Misener, Bountiful, UT (US); Steffan Sowards, Salt Lake City, UT (US); William Robert McLaughlin, Bountiful, UT (US)

(21) Appl. No.: 19/225,986

(22) Filed: Jun. 2, 2025

Related U.S. Application Data
(63) Continuation of application No. 17/689,773, filed on Mar. 8, 2022, now Pat. No. 12,318,149.

(51) Int. Cl. A61B 34/20; A61B 1/00; A61B 5/00; A61B 5/01; A61B 5/283; A61B 5/339; A61M 25/01

(52) U.S. Cl. CPC A61B 34/20; A61B 1/00165; A61B 5/01; A61B 5/283; A61B 5/339; A61B 5/6852; A61B 2034/2061; A61M 25/0158

(57) ABSTRACT

A medical device system includes a stylet with an optical fiber and a console operatively coupled to the stylet. The optical fiber can include an electrically conductive concentric tube configured to transmit electrical signals and a plurality of core fibers within the concentric tube. Each of the plurality of core fibers can include a plurality of sensors, and each the plurality of sensors can be configured to reflect a light signal of a different spectral width based on received incident light and change a characteristic of the reflected light signal based on a condition experienced by the stylet. The console includes one or more processors and a non-transitory computer-readable medium with logic that causes operations including providing an incident light signal to the optical fiber and receiving reflected light signals of different spectral widths of the received incident light by the plurality of sensors.

### PRIORITY

This application is a continuation of U.S. patent application Ser. No. 17/689,773, filed Mar. 8, 2022, now U.S. Pat. No. 12,318,149, which is incorporated by reference in its entirety into this application.

### BACKGROUND

In the past, certain intravascular guidance of medical devices, such as guidewires and catheters for example, have used fluoroscopic methods for tracking tips of the medical devices and determining whether distal tips are appropriately localized in their target anatomical structures. However, such fluoroscopic methods expose patients and their attending clinicians to harmful X-ray radiation. Moreover, in some cases, the patients are exposed to potentially harmful contrast media needed for the fluoroscopic methods.

More recently, electromagnetic tracking systems have been used involving stylets. Generally, electromagnetic tracking systems feature three components: a field generator, a sensor unit and control unit. The field generator uses several coils to generate a position-varying magnetic field, which is used to establish a coordinate space. Attached to the stylet, such as near a distal end (tip) of the stylet for example, the sensor unit includes small coils in which current is induced via the magnetic field. Based on the electrical properties of each coil, the position and orientation of the medical device may be determined within the coordinate space. The control unit controls the field generator and captures data from the sensor unit.

Although electromagnetic tracking systems avoid line-of-sight reliance in tracking the tip of a stylet while obviating radiation exposure and potentially harmful contrast media associated with fluoroscopic methods, electromagnetic tracking systems are prone to interference. More specifically, since electromagnetic tracking systems depend on the measurement of magnetic fields produced by the field generator, these systems are subject to electromagnetic field interference, which may be caused by the presence of many different types of consumer electronics such as cellular telephones. Additionally, electromagnetic tracking systems are subject to signal drop out, depend on an external sensor, and are defined to a limited depth range.

Disclosed herein is an elongate medical device fiber optic sensing system configured to determine a physical state (e.g., three-dimensional shape of the medical device) equipped with an optical fiber that extends distally along a flexible distal tip section. The flexible distal tip section inhibits injury and/or trauma during insertion of the medical device into the patient. The system and device further facilitates obtaining image and electrical signals from the patient body.

### SUMMARY

Briefly summarized, disclosed herein is a medical device including an elongate probe configured for insertion into a patient body, where the elongate probe includes a lumen extending along the elongate probe between a proximal end and a distal end of the elongate probe. The device further incudes an optical fiber disposed within the lumen of the optical fiber, where the optical fiber has one or more of core fibers extending along fibers extending along a longitudinal length of the optical fiber, and where each of the one or more core fibers including a plurality of sensors distributed along the longitudinal length and where each sensor of the plurality of sensors is configured to (i) reflect a light signal of a different spectral width based on received incident light at proximal end, and (ii) change a characteristic of the reflected light signal based on condition experienced by the optical fiber. The elongate probe is operatively coupleable with a console at the proximal end, where the console includes one or more processors and a non-transitory computer-readable medium having stored thereon logic that, when executed by the one or more processors, causes operations that include determining a physical state of the elongate probe within the patient body, and where determining the physical state includes: (i) providing an incident light signal to the optical fiber; (ii) receiving reflected light signals of different spectral widths of the incident light by one or more of the plurality of sensors; and (iii) processing the reflected light signals associated with the one or more of core fibers to determine the physical state. The optical fiber is attached to the elongate probe at an attachment point adjacent the distal end such that the optical fiber is unattached to the elongate probe except at the attachment point.

In some embodiments, the physical state includes one or more of a three-dimensional (3D) shape of the elongate probe, a temperature experienced by the elongate probe, a pressure exerted on the elongate probe, or a fluid flow adjacent the elongate probe.

In some embodiments, the condition experienced by the elongate probe is a strain, and determining the physical state includes processing the reflected light signals associated with the one or more of core fibers to determine the 3D shape. In further embodiments, determining the physical state further includes processing the reflected light signals associated with the one or more of core fibers to determine one or more of the temperature experienced by the elongate probe, the pressure exerted on the elongate probe, or the fluid flow adjacent the elongate probe.

In some embodiments, the optical fiber further includes: (i) one or more illuminating core fibers, where each of the one or more illuminating core fibers is configured to receive illuminating light from the console at the proximal end and project the illuminating light away from the distal end; and (ii) one or more imaging core fibers, where each of the one or more imaging core fibers is configured to receive imaging light at the proximal end and propagate the imaging light along the optical fiber from the distal end to the console.

In some embodiments, the elongate probe further includes a lens at the distal end, and the lens is configured to focus the projected illuminating light.

In some embodiments, the imaging light is an emanation of the projected illuminating light, and the lens is configured to determine a phase shift between the projected illuminating light and the imaging light.

In some embodiments, the elongate probe further includes an electrode located at the distal end, where the electrode is configured to receive an electrical signal from the patient body. According to such embodiments, the optical fiber further includes an electrical conductor extending along the optical fiber, where the electrical conductor is electrically coupled with the electrode, and where the electrical conductor is configured to transmit the electrical signal to the console.

In some embodiments, the elongate probe includes a main probe section coupled with a distal tip section, where the distal tip section has a length between about 20 mm and 80 mm, and in some embodiments, the distal tip section is coupled with the main probe section via one or more of an adhesive, a weld, or a friction fit.

In some embodiments, the distal tip section defines a greater flexibility in bending than the main probe section, and in some embodiments, the distal tip section includes a varying flexibility in bending along the length. In further embodiments, the distal tip section includes a first flexibility in bending adjacent a proximal end of the distal tip section transitioning toward a second flexibility in bending adjacent a distal end of the distal tip section, where the second flexibility in bending is greater than the first flexibility in bending.

In some embodiments, the elongate probe includes a seal configured to prevent fluid exchange between the patient body and the lumen.

In some embodiments, the elongate probe includes an actuator adjacent the proximal end, where the actuator is operatively coupled with the distal tip section, and where the actuator is configured to cause the distal tip section to laterally deflect between a first shape and a second shape based on manipulation of the actuator by a clinician, the second shape different from the first shape.

In some embodiments, the distal tip section includes a shape memory material having a shape transition temperature between about 20° C. and 37° C. In further embodiments, the distal tip section defines a first transition shape below the transition temperature and a second transition shape above the transition temperature, where the second transition shape is different from the first transition shape.

In some embodiments, the medical device is a catheter having the elongate probe disposed with a lumen of the catheter.

Also disclosed herein is a medical device system that generally include a medical device and console. The medical device includes an elongate probe configured for insertion within a patient body, where the elongate probe includes an optical fiber extending along the elongate probe from a distal end to a proximal end. The optical fiber includes one or more of core fibers extending along a longitudinal length of the optical fiber, where each of the one or more core fibers includes a plurality of sensors distributed along the longitudinal length. Each sensor of the plurality of sensors is configured to (i) reflect a light signal of a different spectral width based on received incident light at proximal end, and (ii) change a characteristic of the reflected light signal based on a condition experienced by the optical fiber.

The console is operatively coupled with the medical device, and the console includes one or more processors and a non-transitory computer-readable medium having stored thereon logic that, when executed by the one or more processors, causes operations of the system. The operations include determining a physical state of the elongate probe within the patient body, where determining the physical state includes: (i) providing an incident light signal to the optical fiber; (ii) receiving reflected light signals of different spectral widths of the incident light by one or more of the plurality of sensors; and (iii) processing the reflected light signals associated with the one or more of core fibers to determine the physical state.

The optical fiber is attached to the elongate probe at an attachment point adjacent the distal end such that the optical fiber is unattached to the elongate probe except at the attachment point.

In some embodiments of the system, the physical state includes one or more of a three-dimensional (3D) shape of the elongate probe, a temperature experienced by the elongate probe, a pressure exerted on the elongate probe, or a fluid flow adjacent the elongate probe.

In some embodiments of the system, the condition experienced by the elongate probe is a strain, and determining the physical state includes processing the reflected light signals associated with the one or more of core fibers to determine the 3D shape. In further embodiments, determining the physical state further includes processing the reflected light signals associated with the one or more of core fibers to determine one or more of the temperature experienced by the elongate probe, the pressure exerted on the elongate probe, or the fluid flow adjacent the elongate probe.

In some embodiments of the system, the elongate probe includes a main probe section coupled with a distal tip section, where the distal tip section has a length between about 20 mm and 80 mm.

In some embodiments of the system, the distal tip section includes a varying flexibility in bending along the length, and in further embodiments, the distal tip section includes a first flexibility in bending adjacent a proximal end of the distal tip section transitioning toward a second flexibility in bending adjacent a distal end of the distal tip section, where the second flexibility is greater than the first flexibility.

In some embodiments of the system, the optical fiber further includes: (i) one or more illuminating core fibers, where each of the one or more illuminating core fibers is configured to receive an illuminating light from the console at the proximal end and project the illuminating light away from the distal end; and (ii) one or more imaging core fibers, where each of the one or more imaging core fibers is configured to receive an imaging light at the distal end and propagate the imaging light along the optical fiber from the distal end to the console. According to such embodiments, the operations further include extracting an image of the patient body from the imaging light and causing the image to be portrayed on a display of the system.

In some embodiments of the system, the elongate probe further includes an electrode located at the distal end, where the electrode is configured to receive an electrical signal from the patient body. The optical fiber includes an electrical conductor extending along the optical fiber, where the electrical conductor is electrically coupled between the electrode and the console. According to such embodiments, the operations include extracting an ECG signal from the electrical signal and causing an ECG waveform to be portrayed on a display of the system.

In some embodiments of the system, the elongate probe includes an actuator adjacent the proximal end, where the actuator is operatively coupled with the distal tip section. The actuator is configured to cause the distal tip section to laterally deflect between a first shape and a second shape based on manipulation of the actuator by a clinician.

In some embodiments of the system, the distal tip section includes a shape memory material having a shape transition temperature between about 30° C. and 35° C. In further embodiments, the distal tip section defines a first transition shape below the transition temperature and a second transition shape above the transition temperature, where the second transition shape is different from the first transition shape.

In some embodiments of the system, the medical device is a catheter having the elongate probe disposed with a lumen of the catheter.

These and other features of the concepts provided herein will become more apparent to those of skill in the art in view of the accompanying drawings and following description, which disclose particular embodiments of such concepts in greater detail.

### BRIEF DESCRIPTION OF THE DRAWINGS

Embodiments of the disclosure are illustrated by way of example and not by way of limitation in the figures of the accompanying drawings, in which like references indicate similar elements and in which:

FIG. 1 is an illustrative embodiment of a medical device system including a medical instrument with fiber optic sensing capabilities, in accordance with some embodiments.

FIG. 2 is an exemplary embodiment of a structure of a section of the multi-core optical fiber included within the elongate probe of FIG. 1, in accordance with some embodiments.

FIG. 3A illustrates an embodiment of the elongate probe of FIG. 1, in accordance with some embodiments.

FIG. 3B is a cross sectional view of the elongate probe FIG. 3A, in accordance with some embodiments.

FIGS. 4A-4B are flowcharts of the methods of operations conducted by the medical device monitoring system of FIG. 1 to achieve optic three-dimensional shape sensing, in accordance with some embodiments.

FIG. 5 is a detailed illustration of a distal portion of the elongate probe of FIG. 1, in accordance with some embodiments.

FIG. 6 illustrates another embodiment of the elongate probe having an actuator, in accordance with some embodiments.

FIG. 7 illustrates another embodiment of the elongate probe having a distal tip formed of a shape memory material, in accordance with some embodiments.

### DETAILED DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

With respect to “proximal,” a “proximal portion” or a “proximal end portion” of, for example, a probe disclosed herein includes a portion of the probe intended to be near a clinician when the probe is used on a patient. Likewise, a “proximal length” of, for example, the probe includes a length of the probe intended to be near the clinician when the probe is used on the patient. A “proximal end” of, for example, the probe includes an end of the probe intended to be near the clinician when the probe is used on the patient. The proximal portion, the proximal end portion, or the proximal length of the probe can include the proximal end of the probe; however, the proximal portion, the proximal end portion, or the proximal length of the probe need not include the proximal end of the probe. That is, unless context suggests otherwise, the proximal portion, the proximal end portion, or the proximal length of the probe is not a terminal portion or terminal length of the probe.

With respect to “distal,” a “distal portion” or a “distal end portion” of, for example, a probe disclosed herein includes a portion of the probe intended to be near or in a patient when the probe is used on the patient. Likewise, a “distal length” of, for example, the probe includes a length of the probe intended to be near or in the patient when the probe is used on the patient. A “distal end” of, for example, the probe includes an end of the probe intended to be near or in the patient when the probe is used on the patient. The distal portion, the distal end portion, or the distal length of the probe can include the distal end of the probe; however, the distal portion, the distal end portion, or the distal length of the probe need not include the distal end of the probe. That is, unless context suggests otherwise, the distal portion, the distal end portion, or the distal length of the probe is not a terminal portion or terminal length of the probe.

The term “logic” may be representative of hardware, firmware or software that is configured to perform one or more functions. As hardware, the term logic may refer to or include circuitry having data processing and/or storage functionality. Examples of such circuitry may include, but are not limited or restricted to a hardware processor (e.g., microprocessor, one or more processor cores, a digital signal processor, a programmable gate array, a microcontroller, an application specific integrated circuit (ASIC), etc.), a semiconductor memory, or combinatorial elements.

Additionally, or in the alternative, the term logic may refer to or include software such as one or more processes, one or more instances, Application Programming Interface(s) (API), subroutine(s), function(s), applet(s), servlet(s), routine(s), source code, object code, shared library/dynamic link library (dll), or even one or more instructions. This software may be stored in any type of a suitable non-transitory storage medium, or transitory storage medium (e.g., electrical, optical, acoustical or other form of propagated signals such as carrier waves, infrared signals, or digital signals). Examples of a non-transitory storage medium may include, but are not limited or restricted to a programmable circuit; non-persistent storage such as volatile memory (e.g., any type of random-access memory “RAM”); or persistent storage such as non-volatile memory (e.g., read-only memory “ROM”, power-backed RAM, flash memory, phase-change memory, etc.), a solid-state drive, hard disk drive, an optical disc drive, or a portable memory device. As firmware, the logic may be stored in persistent storage.

The phrases “connected to,” “coupled to,” and “in communication with” refer to any form of interaction between two or more entities, including but not limited to mechanical, electrical, magnetic, electromagnetic, fluid, and thermal interaction. Two components may be coupled to each other even though they are not in direct contact with each other. For example, two components may be coupled to each other through an intermediate component.

Any methods disclosed herein comprise one or more steps or actions for performing the described method. The method steps and/or actions may be interchanged with one another. In other words, unless a specific order of steps or actions is required for proper operation of the embodiment, the order and/or use of specific steps and/or actions may be modified.

References to approximations may be made throughout this specification, such as by use of the term “substantially.” For each such reference, it is to be understood that, in some embodiments, the value, feature, or characteristic may be specified without approximation. For example, where qualifiers such as “about” and “substantially” are used, these terms include within their scope the qualified words in the absence of their qualifiers. For example, where the term “substantially straight” is recited with respect to a feature, it is understood that in further embodiments, the feature can have a precisely straight configuration.

Reference throughout this specification to “an embodiment” or “the embodiment” means that a particular feature, structure, or characteristic described in connection with that embodiment is included in at least one embodiment. Thus, the quoted phrases, or variations thereof, as recited throughout this specification are not necessarily all referring to the same embodiment.

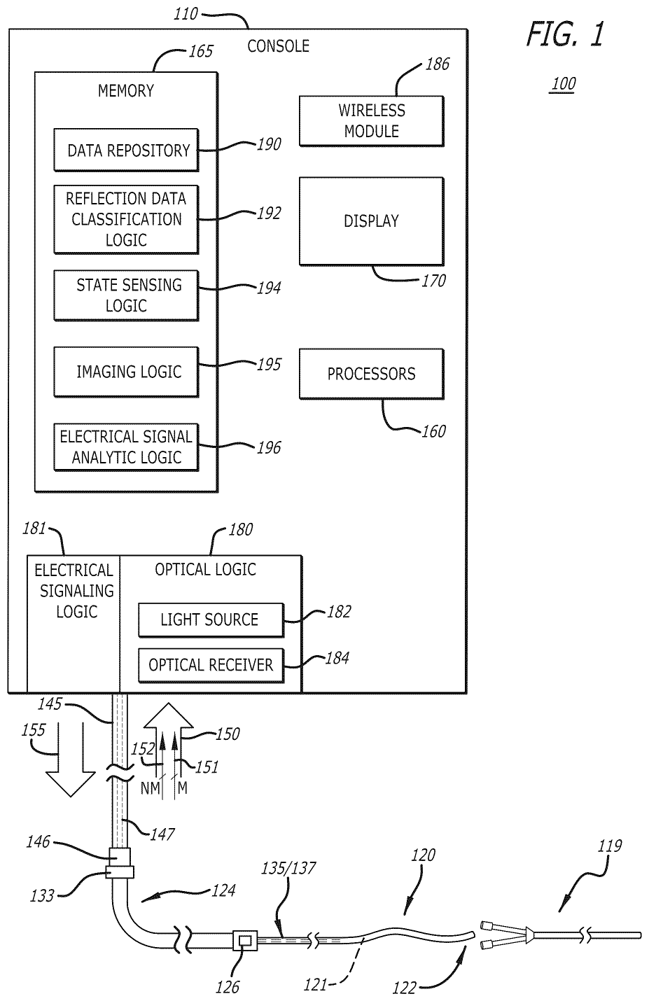

FIG. 1 illustrates an embodiment of a medical instrument placement system including a medical instrument. As shown, the medical instrument placement system (system) 100 generally includes a console 110 and an elongate probe 120 communicatively coupled with the console 110. The elongate probe 120 defines a distal end 122 and includes a console connector 133 at a proximal end 124. The elongate probe 120 includes an optical fiber 135 including multiple core fibers extending along a length of the elongate probe 120 as further described below. The console connector 133 enables the elongate probe 120 to be operably connected to the console 110 via an interconnect 145 including one or more optical fibers 147 (hereinafter, “optical fiber(s)”) and an electrically conductive medium terminated by a single optical/electric connector 146 (or terminated by dual connectors). Herein, the connector 146 is configured to engage (mate) with the console connector 133 to allow for the propagation of light between the console 110 and the elongate probe 120 as well as the propagation of electrical signals from the elongate probe 120 to the console 110.

The elongate probe 120 may be configured to perform any of a variety of medical procedures. As such, the elongate probe 120 may be a component of or employed with a variety of medical instruments/devices 119. In some implementations, the elongate probe 120 may take the form of a guidewire or a stylet for employment within a catheter, for example. The elongate probe 120 may be formed of a metal, a plastic or a combination thereof. The elongate probe 120 includes a lumen 121 extending therealong having an optical fiber 135 disposed therein.

In some implementations, the elongate probe 120 may be integrated into a vascular catheter. Other exemplary implementations include drainage catheters, surgery devices, stent insertion and/or removal devices, biopsy devices, endoscopes, and kidney stone removal devices. In short, the elongate probe 120 may be employed with, or the elongate probe 120 may be a component of, any medical device 119 that is inserted into a patient.

According to one embodiment, the console 110 includes one or more processors 160, a memory 165, a display 170, and optical logic 180, although it is appreciated that the console 110 can take one of a variety of forms and may include additional components (e.g., power supplies, ports, interfaces, etc.) that are not directed to aspects of the disclosure. An illustrative example of the console 110 is illustrated in U.S. Publication No. 2019/0237902, the entire contents of which are incorporated by reference herein. The one or more processors 160, with access to the memory 165 (e.g., non-volatile memory or non-transitory, computer-readable medium), are included to control functionality of the console 110 during operation. As shown, the display 170 may be a liquid crystal display (LCD) integrated into the console 110 and employed as a user interface to display information to the clinician, especially during an instrument placement procedure. In another embodiment, the display 170 may be separate from the console 110. Although not shown, a user interface is configured to provide user control of the console 110.

According to the illustrated embodiment, the content depicted by the display 170 may change according to which mode the elongate probe 120 is configured to operate: optical, TLS, ECG, or another modality. In TLS mode, the content rendered by the display 170 may constitute a two-dimensional or three-dimensional representation of the physical state (e.g., length, shape, form, and/or orientation) of the elongate probe 120 computed from characteristics of reflected light signals 150 returned to the console 110. The reflected light signals 150 constitute light of a specific spectral width of broadband incident light 155 reflected back to the console 110. According to one embodiment of the disclosure, the reflected light signals 150 may pertain to various discrete portions (e.g., specific spectral widths) of broadband incident light 155 transmitted from and sourced by the optical logic 180, as described below.

According to one embodiment of the disclosure, an activation control 126, included on the elongate probe 120, may be used to set the elongate probe 120 into a desired operating mode and selectively alter operability of the display 170 by the clinician to assist in medical device placement. For example, based on the modality of the elongate probe 120, the display 170 of the console 110 can be employed for optical modality-based guidance during probe advancement through the vasculature or TLS modality to determine the physical state (e.g., length, form, shape, orientation, etc.) of the elongate probe 120. In one embodiment, information from multiple modes, such as optical, TLS or ECG for example, may be displayed concurrently (e.g., at least partially overlapping in time).

Referring still to FIG. 1, the optical logic 180 is configured to support operability of the elongate probe 120 and enable the return of information to the console 110, which may be used to determine the physical state associated with the elongate probe 120 within the patient body. Electrical signals, such as ECG signaling, may be processed via an electrical signaling logic 181 that supports receipt and processing of the received electrical signals from the elongate probe 120, (e.g., ports, analog-to-digital conversion logic, etc.). The physical state of the elongate probe 120 may be based on changes in characteristics of the reflected light signals 150 received at the console 110 from the elongate probe 120. The characteristics may include shifts in wavelength caused by strain on certain regions of the core fibers integrated within the optical fiber 135 positioned within or operating as the elongate probe 120, as shown below. As discussed herein, the optical fiber 135 may be comprised of core fibers 137₁-137_{M} (M=1 for a single core, and M≥2 for a multi-core), where the core fibers 137₁-137_{M} may collectively be referred to as core fiber(s) 137. Unless otherwise specified or the instant embodiment requires an alternative interpretation, embodiments discussed herein will refer to an optical fiber 135. From information associated with the reflected light signals 150, the console 110 may determine (through computation or extrapolation of the wavelength shifts) the physical state of the elongate probe 120.

According to one embodiment of the disclosure, as shown in FIG. 1, the optical logic 180 may include a light source 182 and an optical receiver 184. The light source 182 is configured to transmit the incident light 155 (e.g., broadband) for propagation over the optical fiber(s) 147 included in the interconnect 145, which are optically connected to the optical fiber 135 within the elongate probe 120. In one embodiment, the light source 182 is a tunable swept laser, although other suitable light sources can also be employed in addition to a laser, including semi-coherent light sources, LED light sources, etc.

The optical receiver 184 is configured to: (i) receive returned optical signals, namely reflected light signals 150 received from optical fiber-based reflective gratings (sensors) fabricated within each core fiber of the optical fiber 135 deployed within the elongate probe 120, and (ii) translate the reflected light signals 150 into reflection data (from a data repository 190), namely data in the form of electrical signals representative of the reflected light signals including wavelength shifts caused by strain. The reflected light signals 150 associated with different spectral widths may include reflected light signals 151 provided from sensors positioned in the center core fiber (reference) of the optical fiber 135 and reflected light signals 152 provided from sensors positioned in the periphery core fibers of the optical fiber 135, as described below. Herein, the optical receiver 184 may be implemented as a photodetector, such as a positive-intrinsic-negative “PIN” photodiode, avalanche photodiode, or the like.

As shown, both the light source 182 and the optical receiver 184 are operably connected to the one or more processors 160, which governs their operation. Also, the optical receiver 184 is operably coupled to provide the reflection data (from the data repository 190) to the memory 165 for storage and processing by reflection data classification logic 192. The reflection data classification logic 192 may be configured to: (i) identify which core fibers pertain to which of the received reflection data (from the data repository 190) and (ii) segregate the reflection data stored within the data repository 190 provided from reflected light signals 150 pertaining to similar regions of the elongate probe 120 or spectral widths into analysis groups. The reflection data for each analysis group is made available to state sensing logic 194 for analytics.

According to one embodiment of the disclosure, the state sensing logic 194 is configured to compare wavelength shifts measured by sensors deployed in each periphery core fiber at the same measurement region of the elongate probe 120 (or same spectral width) to the wavelength shift at a center core fiber of the optical fiber 135 positioned along central axis and operating as a neutral axis of bending. From these analytics, the state sensing logic 194 may determine the shape the core fibers have taken in three-dimensional space and may further determine the current physical state of the elongate probe 120 in three-dimensional space for rendering on the display 170.

According to one embodiment of the disclosure, the state sensing logic 194 may generate a rendering of the current physical state of the elongate probe 120, based on heuristics or run-time analytics. For example, the state sensing logic 194 may be configured in accordance with machine-learning techniques to access the data repository 190 with pre-stored data (e.g., images, etc.) pertaining to different regions of the elongate probe 120 in which reflected light from core fibers have previously experienced similar or identical wavelength shifts. From the pre-stored data, the current physical state of the elongate probe 120 may be rendered. Alternatively, as another example, the state sensing logic 194 may be configured to determine, during run-time, changes in the physical state of each region of the optical fiber 135 based on at least: (i) resultant wavelength shifts experienced by different core fibers within the optical fiber 135, and (ii) the relationship of these wavelength shifts generated by sensors positioned along different periphery core fibers at the same cross-sectional region of the optical fiber 135 to the wavelength shift generated by a sensor of the center core fiber at the same cross-sectional region. It is contemplated that other processes and procedures may be performed to utilize the wavelength shifts as measured by sensors along each of the core fibers within the optical fiber 135 to render appropriate changes in the physical state of the elongate probe 120, especially to enable guidance of the elongate probe 120 when positioned within the patient and at a desired destination within the body.

The light source 182 and the optical receiver 184 may also be configured to provide illuminating light to the optical fiber 135 and receive imaging light signals from the optical fiber 135, respectively. The imaging logic 195 may be configured to (i) process imaging light signals, (ii) extract/determine an image from the imaging light signals, and (iii) cause the image to be portrayed on the display 170.

The console 110 may further include electrical signaling logic 181 configured to receive one or more electrical signals from the elongate probe 120. The elongate probe 120 is configured to support both optical connectivity as well as electrical connectivity. The electrical signaling logic 181 receives the electrical signals (e.g., ECG signals) from the elongate probe 120 via the conductive medium. The electrical signal logic 196 may process the electrical signals to extract an ECG signal from the electrical signals. The electrical signal logic 196 may further cause an ECG waveform to be portrayed on the display 170.

It is contemplated that other processes and procedures may be performed to utilize the wavelength shifts as measured by sensors along each of the core fibers within the optical fiber 135 to render appropriate changes in the physical state of the probe 120, especially to enable guidance of the probe 120 when positioned within the patient and at a desired destination within the body. For example, wavelength shifts as measured by sensors along one or more of the core fibers may be based on physical states or conditions of the probe 120 other than or in addition to axial strain experienced by the elongate probe 120. Alternative or additional physical states may include one or more of torsional strain, temperature, motion, oscillations, pressure, or fluid flow adjacent the elongate probe.

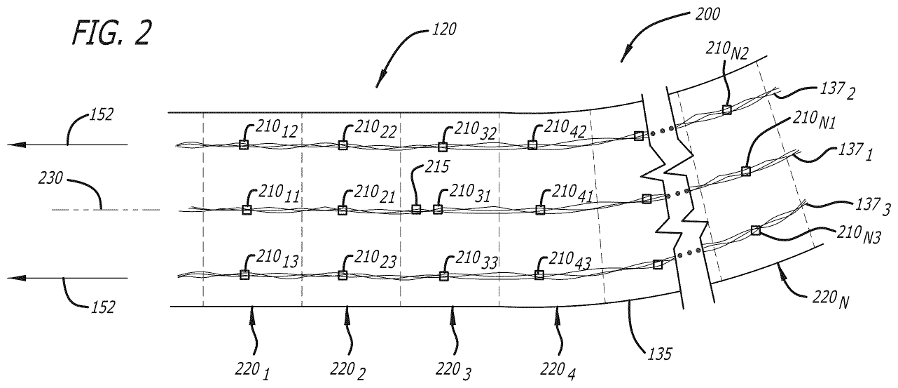

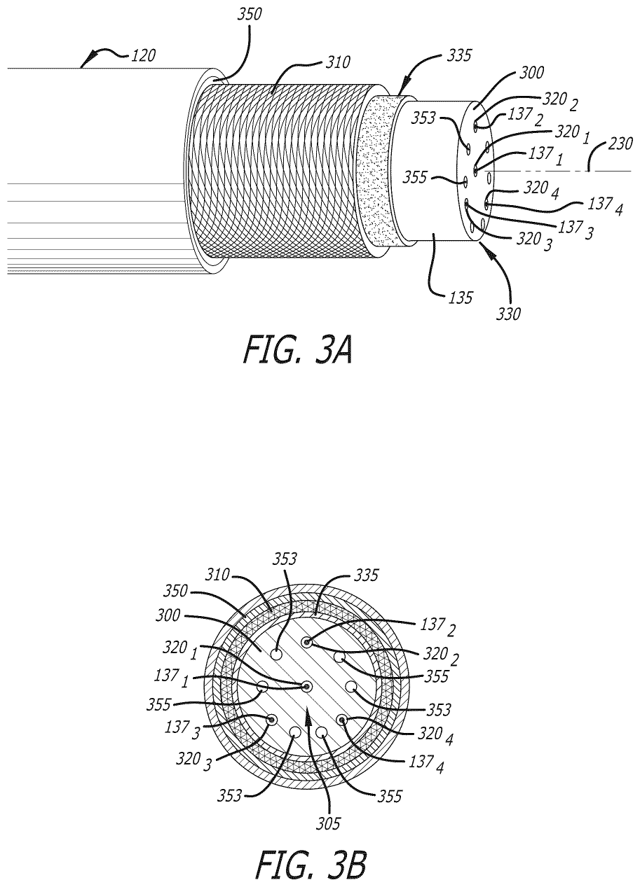

Referring to FIG. 2, an exemplary embodiment of a structure of a section of the multi-core optical fiber included within the elongate probe 120 of FIG. 1 is shown in accordance with some embodiments. The multi-core optical fiber section 200 of the optical fiber 135 depicts certain core fibers 137₁-137_{M} (M≥2, M=4 as shown, see FIG. 3A) along with the spatial relationship between sensors (e.g., reflective gratings) 210₁₁-210_{NM} (N≥2; M≥2) present within the core fibers 137₁-137_{M}, respectively. As noted above, the core fibers 137₁-137_{M} may be collectively referred to as “the core fibers 137.”

As shown, the section 200 is subdivided into a plurality of cross-sectional regions 220₁-220_{N}, where each cross-sectional region 220₁-220_{N} corresponds to reflective gratings 210₁₁-210₁₄ . . . 210_{N1}-210_{N4}. Some or all of the cross-sectional regions 220₁ . . . 220_{N} may be static (e.g., prescribed length) or may be dynamic (e.g., vary in size among the regions 220₁ . . . 220_{N}). A first core fiber 137₁ is positioned substantially along a center (neutral) axis 230 while core fiber 137₂ may be oriented within the cladding of the optical fiber 135, from a cross-sectional, front-facing perspective, to be position on “top” the first core fiber 137₁. In this deployment, the core fibers 137₃ and 137₄ may be positioned “bottom left” and “bottom right” of the first core fiber 137₁. As examples, FIGS. 3A-3B provides illustrations of such.

Referencing the first core fiber 137₁ as an illustrative example, when the elongate probe 120 is operative, each of the reflective gratings 210₁-210_{N} reflects light for a different spectral width. As shown, each of the gratings 210₁ᵢ-210_{Ni} (1≤i≤M) is associated with a different, specific spectral width, which would be represented by different center frequencies of f₁ . . . f_{N}, where neighboring spectral widths reflected by neighboring gratings are non-overlapping according to one embodiment of the disclosure.

Herein, positioned in different core fibers 137₂-137₃ but along at the same cross-sectional regions 220-220_{N} of the optical fiber 135, the gratings 210₁₂-210_{N2} and 210₁₃-210_{N3} are configured to reflect incoming light at same (or substantially similar) center frequency. As a result, the reflected light returns information that allows for a determination of the physical state of the optical fibers 137 (and the elongate probe 120) based on wavelength shifts measured from the returned, reflected light. In particular, strain (e.g., compression or tension) applied to the optical fiber 135 (e.g., at least core fibers 137₂-137₃) results in wavelength shifts associated with the returned, reflected light. Based on different locations, the core fibers 137₁-137₄ experience different types and degree of strain based on angular path changes as the elongate probe 120 advances in the patient.

For example, with respect to the multi-core optical fiber section 200 of FIG. 2, in response to angular (e.g., radial) movement of the elongate probe 120 is in the left-veering direction, the fourth core fiber 137₄ (see FIG. 3A) of the optical fiber 135 with the shortest radius during movement (e.g., core fiber closest to a direction of angular change) would exhibit compression (e.g., forces to shorten length). At the same time, the third core fiber 137₃ with the longest radius during movement (e.g., core fiber furthest from the direction of angular change) would exhibit tension (e.g., forces to increase length). As these forces are different and unequal, the reflected light from reflective gratings 210_{N2} and 210_{N3} associated with the core fiber 137₂ and 137₃ will exhibit different changes in wavelength. The differences in wavelength shift of the reflected light signals 150 can be used to extrapolate the physical configuration of the elongate probe 120 by determining the degrees of wavelength change caused by compression/tension for each of the periphery fibers (e.g., the second core fiber 137₂ and the third core fiber 137₃) in comparison to the wavelength of the reference core fiber (e.g., first core fiber 137₁) located along the neutral axis 230 of the optical fiber 135. These degrees of wavelength change may be used to extrapolate the physical state of the elongate probe 120. The reflected light signals 150 are reflected back to the console 110 via individual paths over a particular core fiber 137₁-137_{M}.

In some embodiments, although not required, that the optical fiber 135 may include sensors 215, where wavelength shifts as measured by the sensors 215 along the optical fiber 135 may be based on physical states or conditions of the probe 120 that include one or more than a temperature experienced by the elongate probe 120, a pressure exerted on the elongate probe 120, or a fluid flow (e.g., blood flow) adjacent the elongate probe 120. The sensors 215 may located along any of the core fibers 137 or along additional core fibers (not shown). In accordance with the sensors 215, the state sensing logic 194 may be configured to determine one or more of the temperature, the pressure, or the fluid flow.

Referring to FIG. 3A, a first exemplary embodiment of the elongate probe 120 of FIG. 1 supporting both an optical and electrical signaling is shown in accordance with some embodiments. Herein, the elongate probe 120 features a centrally located a multi-core optical fiber 135, which includes a cladding 300 and a plurality of core fibers 137₁-137_{M} (M≥2; M=4) residing within a corresponding plurality of lumens 320₁-320_{M}. While the optical fiber 135 is illustrated within four (4) core fibers 137₁-137₄, a greater number of core fibers 137₁-137_{M} (M>4) may be deployed to provide a more detailed three-dimensional sensing of the physical state (e.g., shape, etc.) of the optical fiber 135 and the elongate probe 120 deploying the optical fiber 135.

The optical fiber 135 is encapsulated within a concentric tubing 310 (e.g., braided tubing as shown) positioned over a low coefficient of friction layer 335. The concentric tubing 310, may in some embodiments, feature a “mesh” construction, in which the spacing between the intersecting elements may be selected based on the degree of rigidity/flexibility desired for the elongate probe 120, as a greater spacing may provide a lesser rigidity, and thereby, a more flexible elongate probe 120.

According to this embodiment of the disclosure, as shown in FIGS. 3A-3B, the core fibers 137₁-137₄ include (i) a central core fiber 137₁ and (ii) a plurality of periphery core fibers 137₂-137₄, which are maintained within lumens 320₁-320₄ formed in the cladding 300. According to one embodiment of the disclosure, one or more of the lumen 320₁-320₄ may be configured with a diameter sized to be greater than the diameter of the core fibers 137₁-137₄. By avoiding a majority of the surface area of the core fibers 137₁-137₄ from being in direct physical contact with a wall surface of the lumens 320₁-320₄, the wavelength changes to the incident light are caused by angular deviations in the optical fiber 135 thereby reducing influence of compression and tension forces being applied to the walls of the lumens 320₁-320_{M}, not the core fibers 137₁-137_{M} themselves.

As further shown in FIGS. 3A-3B, the core fibers 137₁-137₄ may include central core fiber 137₁ residing within a first lumen 320₁ formed along the first neutral axis 230 and a plurality of core fibers 137₂-137₄ residing within lumens 320₂-320₄ each formed within different areas of the cladding 300 radiating from the first neutral axis 230. In general, the core fibers 137₂-137₄, exclusive of the central core fiber 137₁, may be positioned at different areas within a cross-sectional area 305 of the cladding 300 to provide sufficient separation to enable three-dimensional sensing of the optical fiber 135 based on changes in wavelength of incident light propagating through the core fibers 137₂-137₄ and reflected back to the console for analysis.

For example, where the cladding 300 features a circular cross-sectional area 305 as shown in FIG. 3B, the core fibers 137₂-137₄ may be positioned substantially equidistant from each other as measured along a perimeter of the cladding 300, such as at “top” (12 o'clock), “bottom-left” (8 o'clock) and “bottom-right” (4 o'clock) locations as shown. Hence, in general terms, the core fibers 137₂-137₄ may be positioned within different segments of the cross-sectional area 305. Where the cross-sectional area 305 of the cladding 300 has a distal tip 330 and features a polygon cross-sectional shape (e.g., triangular, square, rectangular, pentagon, hexagon, octagon, etc.), the central core fiber 137₁ may be located at or near a center of the polygon shape, while the remaining core fibers 137₂-137_{M} may be located proximate to angles between intersecting sides of the polygon shape.

Referring still to FIGS. 3A-3B, the elongate probe 120 may optionally operate as an electrically conductive medium. In some embodiments, the concentric tubing 310 provides mechanical integrity to the optical fiber 135 and operates as a conductive pathway for electrical signals. For example, the concentric tubing 310 may be coupled with a distal tip section of the elongate probe 120. The cladding 300 and the concentric tubing 310, which is positioned concentrically surrounding a circumference of the cladding 300, are contained within the same insulating layer 350. The insulating layer 350 may be a sheath or conduit made of protective, insulating (e.g., non-conductive) material that encapsulates both the cladding 300 and the concentric tubing 310, as shown.

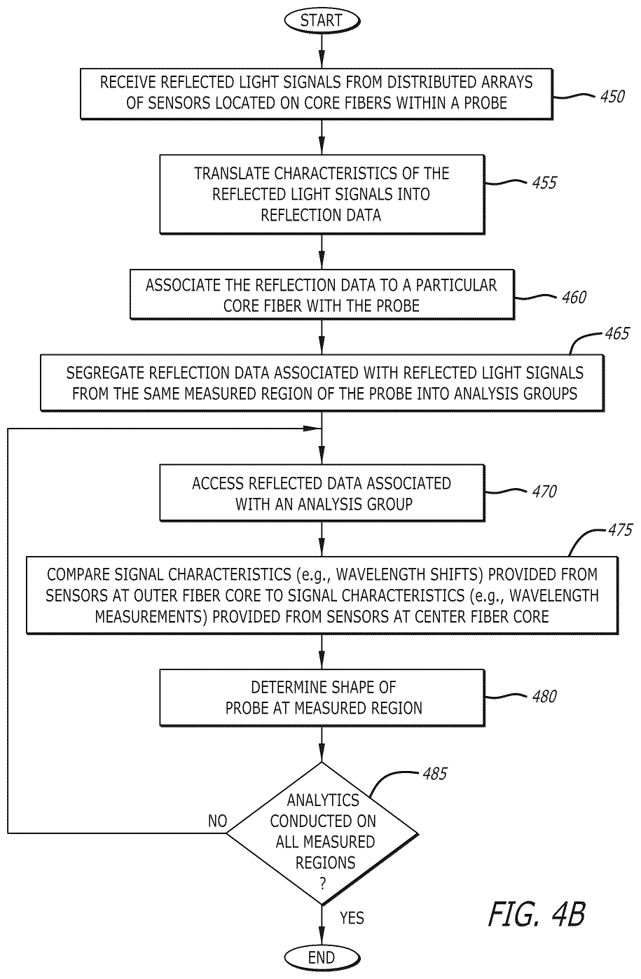

With further reference to FIG. 3B, the elongate probe 120 may optionally include a number of core fibers 353 configured for propagating illuminating light 553 (see FIG. 5) distally along the elongate probe 120 from the console connector 133 to the distal end 122. The illuminating light 553 projects distally away from the distal end 122 (FIG. 1) of the elongate probe 120 to provide visual illumination to an interior of the patient for the purpose of obtaining an image of the patient body, e.g., an image of an interior of a vascular lumen of the patient adjacent the distal end of the elongate probe 120.

The elongate probe 120 may further optionally include a number of core fibers 355 configured for propagating imaging light 555 (see FIG. 5) proximally along the elongate probe 120 from the distal end 122 to the console connector 133. The imaging light 555, as may be defined by the illumination of the patient (e.g., an interior of a vascular lumen of the patient adjacent the distal end of the elongate probe 120), is received by the core fibers 355 at the distal end 122.

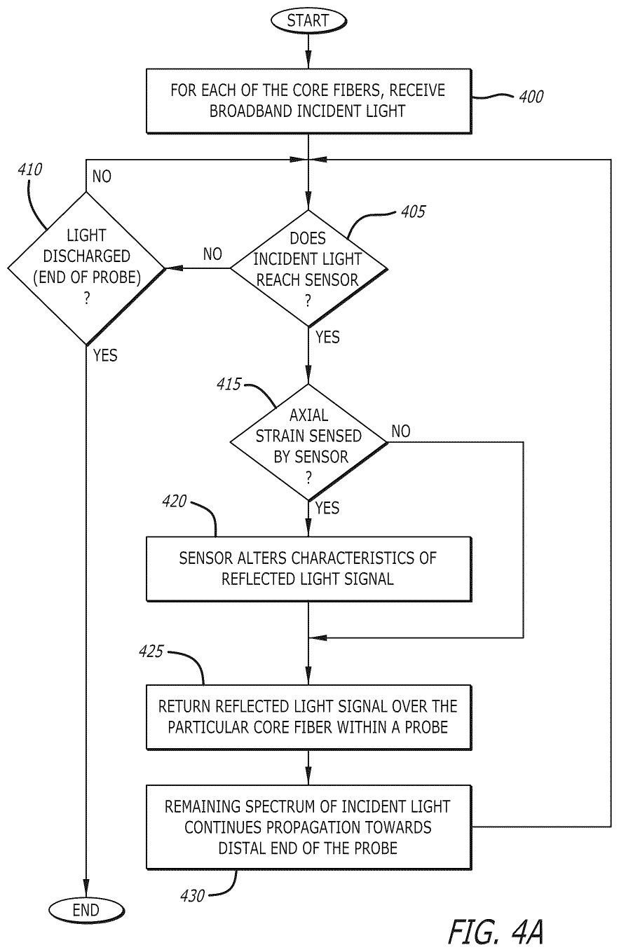

Referring to FIGS. 4A-4B, flowcharts of methods of operations conducted by the medical device system of FIG. 1 to achieve optic three-dimensional shape sensing are shown in accordance with some embodiments. The first micro-lumen is coaxial with the central axis of the probe. The first micro-lumen is configured to retain a center core fiber. Two or more micro-lumen, other than the first micro-lumen, are positioned at different locations circumferentially spaced along the circumferential edge of the probe. For example, two or more of the second plurality of micro-lumens may be positioned at different quadrants along the circumference edge of the probe.

Furthermore, each core fiber includes a plurality of sensors spatially distributed along its length between at least the proximal and distal ends of the probe. This array of sensors is distributed to position sensors at different regions of the core fiber to enable distributed measurements of strain throughout the entire length or a selected portion of the probe. These distributed measurements may be conveyed through reflected light of different spectral widths (e.g., specific wavelength or specific wavelength ranges) that undergoes certain wavelength shifts based on the type and degree of strain, including oscillations of the strain related to motion of the elongate probe 120.

According to one embodiment of the disclosure, as shown in FIG. 4A, for each core fiber, broadband incident light is supplied to propagate through a particular core fiber (block 400). Unless discharged, upon the incident light reaching a sensor of a distributed array of sensors measuring strain on a particular core fiber, light of a prescribed spectral width associated with the first sensor is to be reflected back to an optical receiver within a console (blocks 405-410). Herein, the sensor alters characteristics of the reflected light signal to identify the type and degree of strain on the particular core fiber as measured by the first sensor (blocks 415-420). According to one embodiment of the disclosure, the alteration in characteristics of the reflected light signal may signify a change (shift) in the wavelength of the reflected light signal from the wavelength of the incident light signal associated with the prescribed spectral width. The sensor returns the reflected light signal over the core fiber and the remaining spectrum of the incident light continues propagation through the core fiber toward a distal end of the probe (blocks 425-430). The remaining spectrum of the incident light may encounter other sensors of the distributed array of sensors, where each of these sensors would operate as set forth in blocks 405-430 until the last sensor of the distributed array of sensors returns the reflected light signal associated with its assigned spectral width and the remaining spectrum is discharged as illumination.

Referring now to FIG. 4B, during operation, multiple reflected light signals are returned to the console from each of the plurality of core fibers residing within the corresponding plurality of micro-lumens formed within a probe. In particular, the optical receiver receives reflected light signals from the distributed arrays of sensors located on the center core fiber and the outer core fibers and translates the reflected light signals into reflection data, namely electrical signals representative of the reflected light signals including wavelength shifts caused by strain (blocks 450-455). The reflection data classification logic is configured to identify which core fibers pertain to which reflection data and segregate reflection data provided from reflected light signals pertaining to a particular measurement region (or similar spectral width) into analysis groups (block 460-465).

Each analysis group of reflection data is provided to sensing logic for analytics (block 470). Herein, the sensing logic compares wavelength shifts at each outer core fiber with the wavelength shift at the center core fiber positioned along central axis and operating as a neutral axis of bending (block 475). From this analytics, on all analytic groups (e.g., reflected light signals from sensors in all or most of the core fibers), the sensing logic may determine the shape the core fibers have taken in three-dimensional space, from which the sensing logic can determine the current physical state of the probe in three-dimensional space (blocks 480-485).

FIG. 5 illustrates a detailed view of a distal portion of the elongate probe 120 having the optical fiber 135 disposed within the lumen 121 defined by an annular wall 520. The elongate probe 120 includes a main probe section 521 coupled with a distal tip section 522 at a junction point 526, where the main probe section 521 extends proximally away from the junction point 526. The distal tip section 522 may define a length between about 20 mm and 80 mm, between about 40 mm and 60 mm, or of about 50 mm. In FIG. 5, the distal tip section 522 is shown having a curved shape that may result from a lateral force 505 exerted onto the distal tip section 522 during use. In some embodiments, the distal tip section 522 may define a straight shape in a free state, i.e., in the absence of the lateral force 505.

The main probe section 521 is configured to enable a push-ability of the elongate probe 120 to facilitate insertion of the elongate probe 120 into the patient body, such as during advancement of the elongate probe 120 within a body lumen, for example. More specifically, the main probe section 521 may be sufficiently stiff to facilitate insertion of the elongate probe 120 into the patient body when pushed by a clinician from a location outside the body without the elongate probe 120 buckling within the body.

The distal tip section 522 may be more flexible in bending than the main probe section 521. In other words, the distal tip section 522 may be configured to deflect a greater amount than the main probe section 521 when the lateral force 505 is exerted thereon. The greater flexibility in bending of the distal tip section 522 may facilitate advancement along a body lumen (e.g., a blood vessel, a urethra, etc.) without causing tissue damage or trauma. More specifically, the distal tip section 522 may be configured to deflect due to contact with body tissue, such as a blood vessel wall, for example

In some embodiments, the distal tip section 522 may include a varying flexibility in bending along the distal tip section 522. In other words, the distal tip section 522 may include a first flexibility in bending adjacent the junction point 526 and a second flexibility in bending adjacent the distal end 122, where the second flexibility in bending is greater than the first flexibility in bending. In some embodiments, the first flexibility in bending may gradually transition toward the second flexibility in bending along the length 522A of the distal tip section 522.

The flexibility in bending of the distal tip section 522 may be defined by a structure of the annular wall 520 along the distal tip section 522, a material of the distal tip section 522, or both. Elements of a flexible structure may include one or more of slits, kerfs, bellows, coils, or the like. Flexible materials may include metals (e.g., Nitinol), polymeric materials or elastomeric materials, or any combination thereof.

The distal tip section 522 may include an electrode 560 operatively coupled with (e.g., electrical connected to) the concentric tubing 310 to provide for the transmission of electrical signals from the electrode 560 to the console 110 (see FIG. 1). The electrode 560 may extend along an outside surface of the annular wall 520. For example, the electrode 560 may partially or entirely surround the elongate probe 120. In some embodiments, the electrode 560 may be electrically coupled with the distal tip section 522. As such, the distal tip section 522 may define a portion of the electrical connection between the electrode 560 and the concentric tubing 310.

The elongate probe 120 may optionally include a sealing member 524 which, in some embodiments, may be disposed between the annular wall 520 and the optical fiber 135 as illustrated. The sealing member 524 is configured so that fluid exchange is prevented between the lumen 121 and the patient body. For example, the sealing member 524 may prevent body fluids from entering the lumen 121 and/or migrating proximally along the lumen 121. In some instances, a pressure within the patient adjacent the distal end 122 may be negative (i.e., vacuum). As such, the sealing member 524 may prevent air from migrating distally along the lumen 121 and entering the patient body. The sealing member 524 may take any form suitable for providing the seal. In some embodiments, the sealing member 524 may be an O-ring are similar device. In other embodiments, the sealing member 524 may be formed of a curable substance, such as a curable silicone sealant, for example. The sealing member 524 may be located at any position along the elongate member 120 suitable for sealing off the lumen 121. In some embodiments, the sealing member 524 may form a film covering the distal tip section 622 or a portion thereof.

In the illustrated embodiment, the distal tip section 522 is coupled with main probe section 521 via an adhesive 523. In some embodiments, the adhesive 523 may be an electrically conductive adhesive to facilitate an electrical connection between the distal tip section 522 and the main probe section 521. In alternative embodiments, the distal tip section 522 may be welded to the main probe section 521, where the welding may include ultrasonic welding, spot welding, or TIG welding. In further embodiments, the distal tip section 522 may be attached to the main probe section 521 via a friction fit, such as a press fit, a shrink fit, or a tapered fit, for example.

In some embodiments, the elongate probe 120 may include a lens 550 disposed at the distal end 122. The lens 550 may be attached to the annular wall 520 or the optical fiber 135. The lens 550 may be positioned so that the illuminating light 553 and/or the imaging light passes through the lens 550. In some embodiments, the lens 550 may be configured to focus, disperse, or otherwise cause a phase shift of the illuminating light 553 to optimize or maximize the illumination of an interior of the patient body adjacent the distal end 122 of the elongate probe 120. In some embodiments, the lens 550 may be configured to focus or otherwise cause a phase shift of the imaging light 555 to optimize or otherwise define an image of the illuminated interior of the patient body adjacent the distal end 122. As the imaging light 555 may be an emanation of the illuminating light 553, the lens 550 may be configured to determine a phase shift between illuminating light 553 and imaging light 555.

The optical fiber 135 is attached to the probe 120 (e.g., the annular wall 520) at the distal end 122. More specifically, the optical fiber 135 is attached to the probe 120 only at a single attachment point 525 located adjacent the distal end 122. As such, the optical fiber 135 is unattached to the probe 120 except at the distal end 122. By attaching the optical fiber 135 to the probe 120 only at the distal end 122, the portion of optical fiber 135 extending proximally way from the distal end 122 is allowed to float (e.g., longitudinally slide) within the lumen 121. The floatability of the optical fiber 135 within the lumen 121 may prevent the annular wall 520 from causing undesirable strain along the optical fiber 135, where the undesirable strain may result in a decrease of shape sensing accuracy.

The optical fiber 135 may be attached to the annular wall 520 via an adhesive 527. In some embodiments, the adhesive 527 may be an electrically conductive adhesive to facilitate an electrical connection between the distal tip section 522 and the optical fiber 135 so that electrode 560 is electrically coupled with the concentric tubing 310.

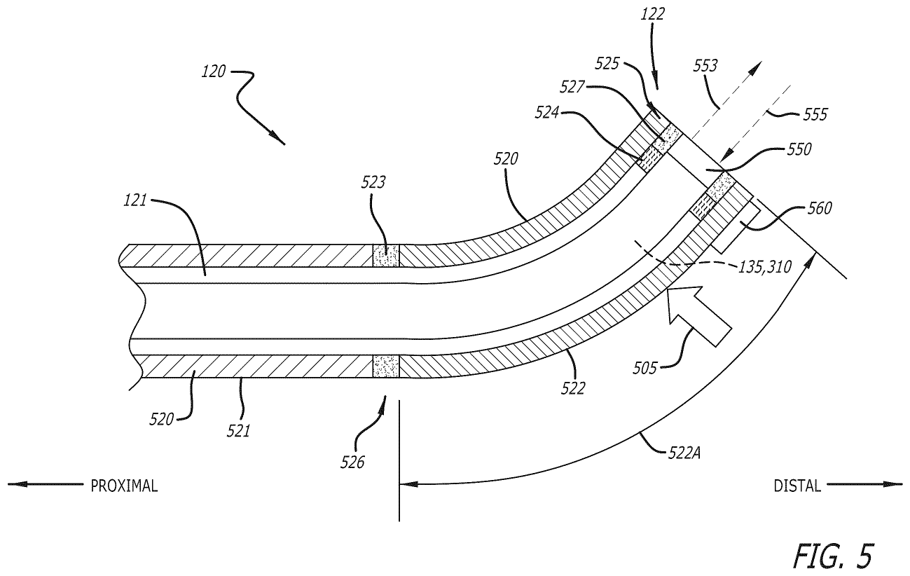

FIG. 6 illustrates another embodiment of an elongate probe that can, in certain respects, include and/or resemble components of the elongate probe 120 described in connection with FIGS. 1-5. It will be appreciated that all the illustrated embodiments may have analogous features. Relevant disclosure set forth above regarding similar features thus may not be repeated hereafter. Moreover, specific features of the elongate probe and related components shown in FIGS. 1-5 may not be shown or identified by a reference numeral in the drawings or specifically discussed in the written description that follows. However, such features may clearly be the same, or substantially the same, as features depicted in other embodiments and/or described with respect to such embodiments. Accordingly, the relevant descriptions of such features apply equally to the features of the elongate probe of FIG. 6. Any suitable combination of the features, and variations of the same, described with respect to the elongate probe and components illustrated in FIGS. 1-5 can be employed with the elongate probe and components of FIG. 6, and vice versa. This pattern of disclosure applies equally to further embodiments depicted in subsequent figures and described hereafter.

The elongate probe 620 includes a steering capability to facilitate insertion within the patient body (e.g., advancement along a lumen of the patient body). Similar to the elongate probe 120, the elongate probe 620 includes a distal tip section 622 that is more flexible that the main probe section 621. The elongate probe 620 also includes a handle 650 having an actuator 651. The actuator 651 is operatively coupled with the distal tip section 622 at the distal end 622A via one or more connecting members 652 (e.g., wires or cables) extending along the elongate probe 620 between the handle 650 and the distal end 622A. The connecting members 652 are coupled between the actuator 651 the distal end 622A such that manipulation of the actuator 651 by a clinician causes fore and/or aft longitudinal displacement of the connecting members 652 which in turn causes a unilateral or bilateral deflection of the distal tip section 622. More specifically, manipulation of the actuator 651 by the clinician may cause the distal tip section 622 to laterally deflect between a first shape and a second shape.

In use, the clinician may deflect the distal tip section 622 based on an image acquired by the elongate probe 620. For example, the clinician may advance the elongate probe 620 along a vasculature of the patient body and identify an optional vasculature pathway within an image acquired by the elongate probe 620. As a result of the identification, the clinician may deflect the distal tip section 622 toward or away from the optional vasculature pathway.

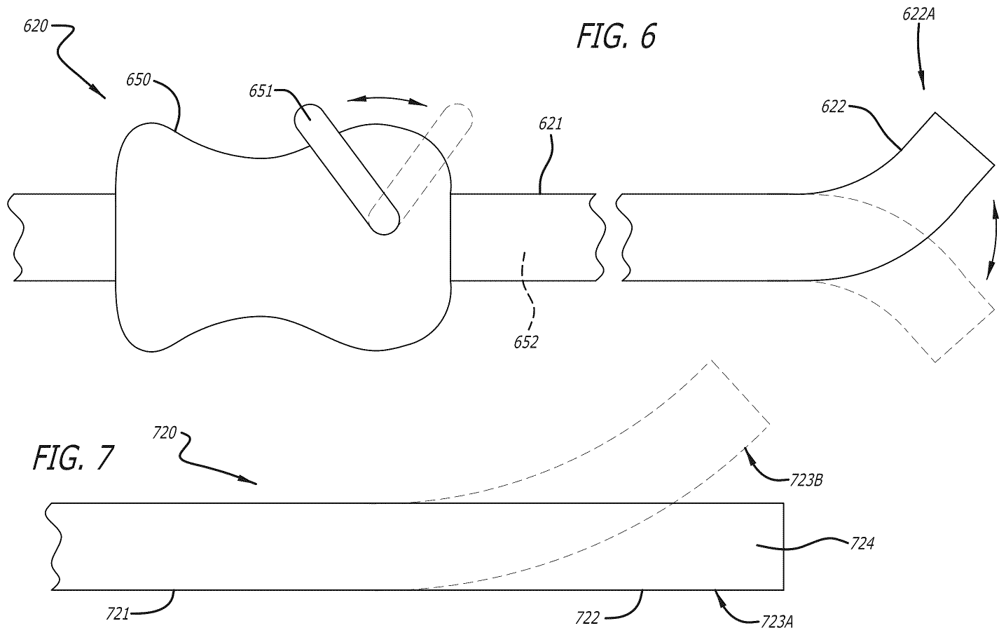

FIG. 7 illustrates another embodiment of an elongate probe 720. The probe 720 generally defines a main probe section 721 and distal tip section 722. The distal tip section 722 is configured to define a first shape 723A and a second shape 723B. The first and second shapes 723A and 723B may exist in a free state, i.e., absent any external force applied to the distal tip section 722. The distal tip section 722 may be formed of a shape memory material 724 (e.g., Nitinol) to facilitate defining the first shape 723A and the second shape 723B.

The distal tip section 722 may be configured to define the first shape 723A at a first point in time and the second shape at a subsequent second point in time. In some embodiments, the distal tip section 722 may (i) define the first shape 723A prior to insertion of the probe 720 within the patient body, and (ii) define the second shape 723B after the probe 720 is inserted within the patient body. In some embodiments, the first shape 723A may be substantially straight to facilitate insertion of the probe 720 into the patient, such as along a vascular access pathway, for example. Similarly, in some embodiments, the second shape 723B may be curved to enable steering of the probe 720 along a vasculature of the patient body.

In some embodiments, the distal tip section 722 may transition from the first shape 723A to the second shape 723B upon or during insertion of the probe 720 into the patient body. As the body temperature may different than the environmental temperature, the distal tip section may be configured to (i) maintain the first shape 723A while the probe 720 is exposed to the environmental temperature, and (ii) transition from the first shape 723A to the second shape 723B when the probe 720 is exposed to the body temperature. As a typical room temperature is about 20° C. and a typical body temperature is about 37° C., the distal tip section 722 may define a transition temperature between 20° C. and 37° C. To account for a range of environmental temperatures, the distal tip section 722 may define a transition temperature that approaches 37° C. As such, the transition temperature of the distal tip section 722 may be between about 20° C. and 37° C., 30° C. and 37° C., or 33° C. and 36° C.

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

What is claimed is:

1. A medical device system, comprising:
a stylet, comprising:
an optical fiber, including:
an electrically conductive concentric tube configured to transmit electrical signals; and
a plurality of core fibers within the concentric tube, each of the plurality of core fibers including a plurality of sensors, each the plurality of sensors configured to:
reflect a light signal of a different spectral width based on received incident light; and
change a characteristic of the reflected light signal based on a condition experienced by the stylet; and
a console operatively coupled to the stylet, the console including one or more processors and a non-transitory computer-readable medium having stored thereon logic that, when executed by the one or more processors, causes operations comprising:
providing an incident light signal to the optical fiber;
receiving reflected light signals of different spectral widths of the received incident light by the plurality of sensors; and
processing the reflected light signals associated with the plurality of core fibers to determine a physical state of the stylet.

2. The medical device system according to claim 1, wherein the physical state of the stylet is selected from the group consisting of a three-dimensional (3D) shape of the stylet, a temperature experienced by the stylet, a pressure exerted on the stylet, a fluid flow adjacent the stylet, and combinations thereof.

3. The medical device system according to claim 2, wherein:
the condition experienced by the stylet is a strain, and
determining the physical state includes processing the reflected light signals associated with the plurality of core fibers to determine the 3D shape.

4. The medical device system according to claim 3, wherein determining the physical state of the stylet further includes processing the reflected light signals associated with the plurality of core fibers to determine the temperature experienced by the stylet, the pressure exerted on the stylet, and/or the fluid flow adjacent the stylet.

5. The medical device system according to claim 1, wherein the stylet further comprises a lens, and wherein the lens is configured to focus a projected illuminating light.

6. The medical device system according to claim 5, wherein an imaging light is an emanation of the projected illuminating light, and wherein the lens is configured to cause a phase shift between the projected illuminating light and the imaging light.

7. The medical device system according to claim 1, wherein the plurality of sensors are distributed along a longitudinal length of the optical fiber.

8. The medical device system according to claim 1, wherein the stylet includes a seal configured to prevent fluid contact with the optical fiber.

9. The medical device system according to claim 1, wherein the stylet includes a main section coupled to a distal tip section, the distal tip section having a length between about 20 mm and 80 mm.

10. The medical device system according to claim 9, wherein the distal tip section is coupled with the main section via one or more of an adhesive, a weld, or a friction fit.

11. The medical device system according to claim 9, wherein the distal tip section defines a greater flexibility in bending than the main section.

12. The medical device system according to claim 9, wherein the distal tip section includes a varying flexibility in bending along the length.

13. The medical device system according to claim 12, wherein the distal tip section includes a first flexibility in bending adjacent a proximal end of the distal tip section transitioning toward a second flexibility in bending adjacent a distal end of the distal tip section, and wherein the second flexibility in bending is greater than the first flexibility in bending.

14. The medical device system according to claim 9, wherein:
the stylet includes an actuator adjacent a proximal end;
the actuator is operatively coupled with the distal tip section; and
the actuator is configured to laterally deflect the distal tip section between a first shape and a second shape based on manipulation of the actuator, the second shape different from the first shape.

15. The medical device system according to claim 9, wherein the distal tip section includes a shape memory material having a shape transition temperature between about 20° C. and 37° C.

16. The medical device system according to claim 15, wherein the distal tip section defines a first transition shape below the shape transition temperature and a second transition shape above the shape transition temperature, the second transition shape different from the first transition shape.

17. The medical device system according to claim 1, further comprising a catheter, wherein the stylet is disposed in a lumen of the catheter.

18. The medical device system according to claim 1, wherein the optical fiber is attached to the stylet at an attachment point adjacent a distal end such that the optical fiber is unattached to the stylet except at the attachment point.
